SEQUENCE LISTING (3 sequences)

SEQ ID NO: 1 - protein, 431 aa, Artificial Sequence
  Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 552 aa, Artificial Sequence
  conbercept
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRLPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 3 - protein, 126 aa, Artificial Sequence
  DARPin MP0112
GSDLGKKLLEAARAGQDDEVRILMANGADVNTADSTGWTPLHLAVPWGHLEIVEVLLKYGADVNAKDFQGWTPLHLAAAIGHQEIVEVLLKNGADVNAQDKFGKTAFDISIDNGNEDLAEILQKAA

(19) United States
(12) Patent Application Publication
(10) Pub. No.: US 2016/0159893 A1
(43) Pub. Date: Jun. 9, 2016

(54) Use of a VEGF Antagonist in Treating Retinopathy of Prematurity

(71) Applicants: Gabriela BURIAN, Basel (CH); Sergey AKSENOV, Winchester, MA (US)

(72) Inventors: Gabriela BURIAN, Basel (CH); Sergey AKSENOV, Winchester, MA (US)

(21) Appl. No.: 14/903,435

(22) PCT Filed: Jul. 10, 2014
(86) PCT No.: PCT/IB2014/063003

§ 371 Date: Jan. 7, 2016

Related U.S. Application Data
(60) Provisional application No. 61/845,073, filed on Jul. 11, 2013.

(51) Int. Cl. C07K 16/22; A61B 18/02; A61F 9/008

(52) U.S. Cl. CPC C07K 16/22; A61F 9/00823; A61B 18/02; A61K 2039/505

(57) ABSTRACT

The present invention relates to the use of a VEGF antagonist in the treatment of retinal neovascular disorders in infants. In particular, the invention provides a method for treating an infant having retinopathy of prematurity (ROP), wherein said method comprises administering to the eye of an infant a VEGF antagonist that either does not enter or is rapidly cleared from the systemic circulation. The term “infant” is typically used to refer to young children from birth up to the age of 12 months. The VEGF antagonist may be administered intravitreally, e.g. through injection, or topically, e.g. in form of eye drops.

### TECHNICAL FIELD

This invention is in the field of treating retinal disorders in infants.

### BACKGROUND ART

Retinal neovascularisation together with retinal detachment is the hallmark of retinopathy of prematurity (ROP). 50-65% of premature infants weighing less than 1250 g at birth suffer from this form of retinopathy. ROP is a leading cause of childhood blindness worldwide.

Depending on the severity of ROP, the standard of care ranges from watchful waiting to surgical intervention. The main treatment goal is to restore retinal function and preserve vision. No treatment is typically recommended for infants with mild or moderate abnormal blood vessel growth as in the majority of these infants, the disease does not progress further and resolves on its own over time.

More advanced stages of ROP, which are associated with severely abnormal retinal neovascularisation often followed by partial or complete detachment of the retina, require therapeutic intervention, typically by cryotherapy or laser photocoagulation therapy (LPT). Both forms of therapy result in the destruction of at least part of the peripheral retina.

In the recently reported BEAT-ROP study, intravitreal bevacizumab monotherapy was compared with conventional LPT in infants with stage 3+ ROP (Mintz-Hittner et al. (2011) N Engl. Med 364(7):603-15). A significant benefit for zone I but not zone II disease was observed with infants receiving intravitreal bevacizumab. Peripheral retinal vessels continued to develop after treatment with intravitreal bevacizumab, whereas conventional laser photocoagulation therapy resulted in permanent destruction of the peripheral retina. The BEAT-ROP study (and most other subsequent studies) adopted half the adult dose of bevacizumab for their intravitreal injections in premature infants.

Unlicensed bevacizumab is widely used for the treatment of age-related macular degeneration and other chorioretinal pathologies in adults, although it was developed for systemic use in treating colon cancer. It is often difficult to predict how a drug successfully used in adults will behave in a paediatric population, especially in younger children (0-12 years). Since bevacizumab is usually administered intravitreally to treat ocular diseases, some concerns have been voiced that a small amount of an antibody VEGF antagonist could enter the brain where it might interfere with a child's normal brain development (Sivaprasad et al. (2008) Br J Ophthalmol. 92:451-54). Potential concerns have also been raised with respect to the systemic exposure to an antibody VEGF antagonist when treating children (Lyall et al. (2010) Eye 24: 1730-31).

It is thus an object of the invention to provide further and improved treatments for retinal disorders in infants that address at least some of the current concerns referred to above.

### DISCLOSURE OF THE INVENTION

The present invention relates to the use of a VEGF antagonist in the treatment of retinal neovascular disorders in infants. In particular, the invention provides a method for treating a premature infant having retinopathy of prematurity (ROP), wherein said method comprises administering to the eye of the infant a VEGF antagonist that either does not enter or is rapidly cleared from the systemic circulation. The VEGF antagonist may be administered intravitreally, e.g. through injection, or topically, e.g. in form of eye drops.

### The Patient

The invention relates to the treatment of infants suffering from a retinal neovascular disorder. The term “infant” is used to refer to young children from birth up to the age of 12 months.

In a specific aspect of the invention, the invention relates to the treatment of ROP in premature or pre-term infants. The terms “premature infant” and “pre-term infant”typically refer to an infant born at less than 37 weeks gestational age.

In some instances, treatment criteria will depend on the postmenstrual age of the infant treated for ROE. The postmenstrual age is the time elapsed between the first day of the last menstrual period and birth (gestational age) plus the time elapsed after birth (chronological age). Postmenstrual age is usually described in number of weeks. For example, a preterm infant born at a gestational age of 33 weeks who is currently 10 weeks old (chronological age) has a postmenstrual age of 43 weeks.

### VEGF Antagonists

VEGF is a well-characterised signal protein which stimulates aniogenesis. Two antibody VEGF antagonists have been approved for human use, namely ranibizumab (Lucentis®) and bevacizumab (Avastin®).

While ranibizumab and bevacizumab have similar clearance rates from the eye into the blood stream, ranibizumab is excreted rapidly from the systemic circulation, whereas bevacizumab is retained and can suppress systemic VEGF levels for several weeks. More specifically, ranibizumab has a short systemic half-life of about 2 hours, whereas bevacizumab has a systemic half-life of about 20 days. In a developing organism like an infant, this prolonged systemic VEGF suppression may have unwanted side effects on the normal development.

Therefore, in one aspect, the invention relates to the use of a VEGF antagonist in the treatment of a retinal neovascular disorder in an infant wherein the VEGF antagonist either does not enter or is rapidly cleared from the infant's systemic circulation. In accordance with the invention, clearance of the VEGF antagonist may be sufficiently rapid when the systemic half-life of the VEGF antagonist is between 7 days and about 1 hour. Preferably, the systemic half-life of the VEGF antagonist of the invention is less than 7 days, more preferably less than 1 day, most preferably less than 3 hours. A preferred antibody VEGF antagonist is ranibizumab.

As an alternative, the VEGF antagonist is a non-antibody VEGF antagonist. Non-antibody antagonists include e.g. immunoadhesins. One such immunoadhesin with VEGF antagonist activity is aflibercept (Eylea®), which has recently been approved for human use and is also known as VEGF-trap (Holash et al. (2002) PNAS USA 99:11393-98; Rielf & Miller (2007) Clin Cancer Res 13:4623-7s). Aflibercept has a systemic half-life of around 5-6 days and is the preferred non-antibody VEGF antagonist for use with the invention. Aflibercept is a recombinant human soluble VEGF receptor fusion protein consisting of portions of human VEGF receptors 1 and 2 extracellular domains fused to the Fe portion of human IgG1. It is a dimeric glycoprotein with a protein molecular weight of 97 kilodaltons (kDa) and contains glycosylation, constituting an additional 15% of the total molecular mass, resulting in a total molecular weight of 115 kDa. It is conveniently produced as a glycoprotein by expression in recombinant CHO K1 cells. Each monomer can have the following amino acid sequence (SEQ ID NO: 1):

SDTGRPFVEMYSEIPEIIHMTEGRELVIPCRVTSPNITVTLKKFPLDTL
IPDGKRIIWDSRKGFIISNATYKEIGLLTCEATVNGHLYKTNYLTHRQT
NTIIDVVLSPSHGIELSVGEKLVLNCTARTELNVTVGIDFNWEYPSSKH
QHKKLVNRDLKTQSGSEMKKFLSTLTIDGVTRSDQGLYTCAASSGLMTK
KNSTFVRHEKDKTHTCPPCPAPELLGGPSVFLFPPKPKDTLMISRTPEV
TCVVVDVSHEDPEVKFNWYVDGVEVHNAKTKPREEQYNSTYRVVSVLTV
LHQDWLNGKEYKCKVSNKALPAPIEKTISKAKGQPREPVQVTLPPSRDE
LTKNQVSLTCLVKGFYPSDIAVEWESNGQPENNYKTTPPVLDSDGSFFL
YSKLTVDKSRWQQGNVFSCSVMHEALHNHYTQKSLSLSPG

and disulfide bridges can be formed between residues 30-79, 124-185, 246-306 and 352-410 within each monomer, and between residues 211-211 and 214-214 between the monomers.

Another non-antibody VEGF antagonist immunoadhesin currently in pre-clinical development is a recombinant human soluble VEGF receptor fusion protein similar to VEGF-trap containing extracellular ligand-binding domains 3 and 4 front VEGFR2/KDR, respectively, and domain 2 from VEGFR1/Flt-1; these domains are fused to a human IgG Fe protein fragment (Li et al. (2011) Molecular Vision 17:797-803). This antagonist binds to isoforms VEGF-A, VEGF-B and VEGF-C. The molecule is prepared using two different production processes resulting in different glycosylation patterns on the final proteins. The two glycoforms are referred to as KH902 (conbercept) and KH906. The fusion protein can have the following amino acid sequence (SEQ ID NO:2):

MVSYWDTGVLLCALLSCLLLTGSSSGGRPFVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTAR
TELNVGIDFNWEYPSSKHQHKKLVNRDLKTQSGSEMKKFLSTLTIDGVTR
SDQGLYTCAASSGLMTKKNSTFVRVHEKPFVAFGSGMESLVEATVGERVR
LPAKYLGYPPPEIKWYKNGIPLESNHTIKAGHVLTIMEVSERDTGNYTVI
LTNPISKEKQSHVVSLVVYVPPGPGDKTHTCPLCPAPELLGGPSVFLFPP
KPKDTLMISRTPEVTCVVVDVSHEDPEVKFNWYVDGVEVHNAKTKPREEQ
YNSTYRVVSVLTVLHQDQLNGKEYKCKVSNKALPAPIEKTISKAKGQPRE
PQVYTLPPSRDELTKNQVSLTCLVKGFYPSDIAVEWESGNQPENNYKATP
PVLKSDGSFFLYSKLTVDKSRWQQGNVFSCSVMHEALHNHYTQKSLSLSP
GK

and, like VEGF-trap, can be present as a dimer. This fusion protein and related molecules are further characterized in EP1767546.

Other non-antibody VEGF antagonists include antibody mimetics (e.g. Affibody® molecules, affilins, affitins, anticalins, avimers, Kunitz domain peptides, and monobodies) with VEGF antagonist activity. Due to their small size, antibody mimetics are typically cleared from the circulation rapidly (within minutes to hours). Pegylation is one way used to extend local and systemic half-life.

Therefore the term “non-antibody VEGF antagonists” includes recombinant binding proteins comprising an ankyrin repeat domain that binds VEGF-A and prevents it from binding to VEGFR-2. One example for such a molecule is DARPin® MP0112. The ankyrin binding domain may have the following amino acid sequence (SEQ ID NO: 3):

GSKLGKKLLEAARAGQDDEVRILMANGADVNTADSTGWTPLHLAVPWGH
LEIVEVLLKYGADVNAKDFQGWTPLHLAAAIGHQEIVEVLLKNGADVNA
QDKFGKTAFDISIDNGNEDLAEILQKAA

Recombinant binding proteins comprising an ankyrin repeat domain that binds VEGF-A and prevents it from binding to VEGFR-2 are described in more detail in WO2010/060748 and WO2011/135067. Pegylation extends the systemic half-life of DARPins® to 1-3 days.

Further specific antibody mimetics with VEGF antagonist activity are the 40 kD pegylated Anticalin® PRS-050 (Mross et al. (2011) Molecular Cancer Therapeutics 10: Supplement 1, Abstract A212) and the monobody pegdinetanib (also referred to as Angiocept or CT-322, see Dineen et al. (2008) BAC Cancer 8:352).

The afore-mentioned non-antibody VEGF antagonist may be modified to further improve their pharmacokinetic properties. For example, a non-antibody VEGF antagonist may be chemically modified, mixed with a biodegradable polymer or encapsulated into microparticies to increase intravitreal retention of and reduce systemic exposure to the non-antibody VEGF antagonist.

Variants of the above-specified VEGF antagonists that have improved characteristics for the desired application may be produced by the addition or deletion of amino acids. Ordinarily, these amino acid sequence variants will have an amino acid sequence having at least 60% amino acid sequence identity with the amino acid sequences of SEQ ID NO: 1, SEQ ID NO: 2 or SEQ ID NO: 3, preferably at least 80%, more preferably at least 85%, more preferably at least. 90%, and most preferably at least 95%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%. Identity or homology with respect to this sequence is defined herein as the percentage of amino acid residues in the candidate sequence that are identical with SEQ ID NO: 1, SEQ ID NO: 2 or SEQ ID NO: 3, after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a standard scoring matrix such as PAM 250 can be used in conjunction with the computer program (see Dayhoff et al. (1978) Atlas of Protein Sequence and Structure, vol. 5, supp. 3). For example, the percent identity can then be calculated as: the total number of identical matches multiplied by 100 and then divided by the sum of the length of the longer sequence within the matched span and the number of gaps introduced into the shorter sequences in order to align the two sequences.

If a non-antibody VEGF antagonist is used in practising the invention, the non-antibody VEGF antagonist binds to VEGF via one or more protein domain(s) that are not derived from the antigen-binding domain of an antibody. The non-antibody VEGF antagonist is preferably proteinaceous, but may include modification that are non-proteinaceous (e.g., pegylation, glycosylation). In some embodiments of the invention, the VEGF antagonist of the invention preferably does not comprise the Fc portion of an antibody as the presence of the Fc portion in some instances increases the half-life of the VEGF antagonist and extends the time the VEGF antagonist is present in circulation.

### Pegylation

Due to their small size, antibody mimetics are typically cleared from the circulation rapidly (within minutes to hours). Thus, in some embodiments of the invention, in particular where the VEGF antagonist is an antibody mimetic, one or more polyethylene glycol moieties may be attached at different positions in the VEGF antagonist molecule.

Such attachment may be achieved by reaction with amines, thiols or other suitable reactive groups. The thiol group may be present in a cysteine residue; and the amine group may be, for example, a primary amine found at the N-terminus of the polypeptide or an amine group present in the side chain of an amino acid, such as lysine or arginine.

Attachment of polyethylene glycol (PEG) moieties (pegylation) may be site-directed. For instance, a suitable reactive group may he introduced into the VEGF antagonist to create a site where pegylation can occur preferentially. For example, such a VEGF antagonist antibody mimetic (e.g. DARPin® MP0112) may be modified to include a cysteine residue at a desired position, permitting site directed pegylation on the cysteine, for example by reaction with a PEG derivative carrying a maleimide function. Alternatively, a suitable reactive group may already originally be present in the VEGF antagonist.

The PEG moiety may vary widely in molecular weight (i.e. from about 1 kDa to about 100 kDa) and may he branched or linear. Preferably, the PEG moiety has a molecular weight of about 1 to about 50 kDa, preferably about 10 to about 40 kDa, even more preferably about 15 to about 30 kDa, and most preferably about 20 kDa. For example, addition of a PEG moiety of 20 kDa has been shown to extend the half-life of DARPin® in circulation to up to 20 hours, while larger PEG moieties of 40 to 60 kDa in size increased circulatory half-life to about 50 hours.

### Dosing

Ranibizumab is typically administered to adults intravitreally at a dose of 0.5 mg in a 50 μl volume. Aflibercept is also administered via intravitreal injection, and the typical adult dose is 2 mg (suspended in 0.05 ml buffer comprising 40 mg/ml in 10 mM sodium phosphate, 40 mM sodium chloride, 0.03% polysorbate 20, and 5% sucrose, pH 6.2).

However, the normal dose and/or volume may be reduced for the VEGF antagonist treatment of smaller children and especially for the VEGF antagonist treatment of infants due the reduced intravitreal volume of their eyes and the increased risk associated with systemic VEGF antagonist exposure. Typically, an adult VEGF antagonist preparation is used to treat children, and the dose is simply adjusted by reducing the volume administered to the child. For example, the BEAT-ROP study (and most other subsequent studies with premature infants suffering from ROP) adopted half the adult bevacizumab dose for their injections in premature infants.

However, it is not always practical to simply reduce the volume to adjust the dose of VEGF antagonist that is administered. Therefore, in one embodiment, only the VECF antagonist dose is reduced (e.g. to reduce systemic VEGF antagonist exposure), while the administered volume is kept the same. Dose reduction can be achieved by diluting an adult VEGF antagonist formulation through the addition of a sterile, buffered solution (ideally the same buffer in which the VEGF antagonist is provided in the adult formulation). In other embodiments, the same VEGF antagonist dose is administered, but in a reduced volume (to account for the smaller size of the eye in infants). Preferably, both the VEGF antagonist dose and the volume in which it is administered are reduced. For example, the dose and the volume may be reduced proportionally to the reduced intravitreal volume of the eye according to the age of the child to be treated in order to maintain the same ocular concentration that have been found to be efficacious in adults. For example, a 6 mg/ml formulation of ranibizumab is particularly suitable to provide doses and volumes adapted for different age and patient groups (e.g. 0.06 mg, 0.12 mg, 0.18 mg and 0.24 mg in 10 μl, 20 μl, 30 μl and 40 μl, respectively). Also, a 10 mg/ml formulation of ranibizumab is suitable to provide doses and volumes adapted for different age and patient groups (e.g. 0.05 mg, 0.10 mg, 0.15 mg and 0.20 mg in 5 μl, 10 μl, 15 μl and 20 μl, respectively).

Smaller volumes are sometimes harder to manage and may result in greater variation of the amount of VEGF antagonist actually administered to a patient. Therefore in some embodiments, the dose is reduced without reducing the volume that is used to administer the VEGF antagonist.

Preferably, the dose for treating an infant with a VEGF antagonist in accordance with the invention is less than 50% of the dose typically administered to an adult (e.g. less than 40%, preferably less than 30%, more preferably less than 20%). Reducing the dose proportionally to the reduced intravitreal volume of the eye of an infant is typically not sufficient to prevent systemic VEGF antagonist exposure levels that exceed those that were found to be safe in the adult population. Systemic exposure is correlated to the body weight of the subject. Therefore, when choosing, specific doses for the administration to infants, the possibility of underexposure relative to the reference adult vitreal exposure (decreased efficacy) needs to be balanced against the increased serum exposure (increased risk). Hence, in accordance with the invention, the dose administered to an infant is reduced further than what would be dictated by a proportional reduction relative to the reduced intravitreal volume of the infant's eye in order to maintain safe systemic VEGF antagonist exposure levels. Generally, the dose of a VEGF antagonist administered to an infant is about 10% to about 25% of the typical adult dose. For example, the dose may be reduced to about one fourth to about one eighth of the typical adult dose (e.g. about one fifth, one sixth, or one seventh of the typical adult dose).

In accordance with the invention, the volume in which the VEGF antagonist dose is administered to the infant is less than 50% of the volume typically administered to an adult (e.g. less than 40%, preferably less than 30%, more preferably less than 20%). Generally, the volume of a VEGF antagonist administered to an infant is about 10% to about 25% of the volume typically administered to an adult. For example, the volume typically administered to an adult may be reduced to about one fourth to about one eighth for administration to an infant (e.g. to about one fifth, one sixth, or one seventh of the adult volume typically administered).

In a specific embodiment of the invention, lower doses of ranibizumab can achieve similar results in controlling ROP and do not cause systemic VEGF suppression to the same extent as prior art treatments. For the treatment of ROP in infants by intravitreal ranibizumab injections, doses of less than 0.25 mg are preferred. In one embodiment, 0.05-0.25 mg ranibizumab is administered per dose. in a preferred embodiment, 0.1-0.2 mg ranibizumab is administered per dose. For example, the ranibizumab dose can be reduced to 0.20 mg, preferably to 0.18 mg, preferably 0.12 mg, more preferably 0.06 mg by administering 30 μl, 20 μl or 10 μl of a standard 6 mg/ml ranibizumab solution. In some instances, larger doses may be necessary to achieve efficacy (e.g. 0.25 mg ranibizumab in 25 μl, or up to 0.24 mg ranibizumab in 40 μl). Alternatively, infants suffering from ROP may receive 0.15 mg, preferably 0.1 mg, more preferably 0.075 mg ranibizumab. To achieve these does; 15 μl, 10 μl and 7.5 μl of a standard 10 mg/ml ranibizumab solution is administered.

### Administration

The VEGF antagonist of the invention will generally be administered to the patient via intravitreal injection. Administration in aqueous form is usual, with a typical volume of 5-50 μl e.g. 7.5 μl, 10 μl, 15 μl, 20 μl, 25 μl, or 30 μl. Injection can be performed with a 30-gauge×½-inch (0.3 mm×13 mm) needle.

In one aspect of the invention, the VEGF antagonist is provided in a pre-filled sterile syringe ready for administration. Preferably, the syringe has low silicone content. More preferably, the syringe is silicone free. The syringe may be made of glass. Using a pre-filled syringe for delivery has the advantage that any contamination of the sterile VEGF antagonist solution prior to administration can be avoided. Pre-filled syringes also provide easier handling for the administering ophthalmologist.

In accordance with the invention, a pre-filled syringe will contain a suitable dose and volume of a VEGF antagonist of the invention. Typically, both the dose and the volume in the pre-filled syringe is less than 50% of the typical dose and volume of a VEGF antagonist administered to an adult. A typical volume of VEGF antagonist in the pre-filled syringe is 5-50 μl, e.g. 7.5 μl , 10 μl, 15 μl, 20 μl, 25 μl, or 30 μl. For example, a prefilled syringe may contain a 6 mg/ml formulation of ranibizumab (e.g. comprising 0.06 mg, 0.12 mg, 0.18 mg and 0.24 mg in 10 μl, 20 μl, 30 μl and 40 μl, respectively). Alternatively, a pre-filled syringe may contain a 10 mg/ml formulation of ranibizumab (e.g. comprising 0.2 mg, 0.15 mg, 0.1 mg or 0.075 mg in 20 μl, 15 μl, 10 μl and 7.5 μl, respectively).

In a preferred embodiment, a pre-filled low-dose syringe in accordance with the invention has a nominal maximal fill volume of 0.2 ml and is specifically adapted to accurately dispense volumes below 50 μl.

### Slow-Release Formulations

The VEGF antagonist may be provided in a slow-release formulation. Slow-release formulations are typically obtained by mixing a therapeutic agent with a biodegradable polymer or encapsulating it into microparticles. By varying the manufacturing conditions of polymer-based delivery compositions, the release kinetic properties of the resulting compositions can be modulated. Addition of a polymeric carrier also reduces the likelihood that any intravitreal administered VEGF antagonist enters the circulation or reaches the developing brain of a child.

A slow-release formulation in accordance with the invention typically comprises a VEGF antagonist, a polymeric carrier, and a release modifier for modifying a release rate of the VEGF antagonist from the polymeric carrier. The polymeric carrier usually comprises one or more biodegradable polymers or co-polymers or combinations thereof. For example, the polymeric carrier may be selected from poly-lactic acid (PLA), poly-glycolic acid (PGA), poly-lactide-co-glycolide (PLGA), polyesters, poly (orthoester), poly(phosphazine), poly(phosphate ester), polycaprolactones, or a combination thereof. A preferred polymeric carrier is PLGA. The release modifier is typically a long chain fatty alcohol, preferably comprising from 10 to 40 carbon atoms. Commonly used release modifiers include capryl alcohol, pelargonic alcohol, capric alcohol, lauryl alcohol, myristyl alcohol, cetyl alcohol, palmitoleyl alcohol, stearyl alcohol, isostearyl alcohol, elaidyl alcohol, oleyl alcohol, linoleyl alcohol, polyunsaturated elaidolinoleyl alcohol, polyunsaturated linolenyl alcohol, elaidolinolenyl alcohol, polyunsaturated ricinoleyl alcohol, arachidyl alcohol, behenyl alcohol, erucyl alcohol, lignoceryl alcohol, ceryl alcohol, montanyl alcohol, cluytyl alcohol, myricyl alcohol, melissyl alcohol, and geddyl alcohol.

In a particular embodiment, the VEGF antagonist is incorporated into a microsphere-based sustained release composition. The microspheres are preferably prepared from PLGA. The amount of VEGF antagonist incorporated in the microspheres and the release rate of the VEGF antagonist can be controlled by varying the conditions used for preparing the microspheres. Processes for producing such slow-release formulations are described in US 2005/0281861 and US 2008/0107694.

The need and extent for dose and release-rate adjustment for a slow-release formulation suitable for administration to infants can he assessed using the ocular and systemic exposure models described herein.

### Treatment Regimens

In accordance with the invention, the VEGF antagonist is administered one or more times initially and then re-administered “as needed” depending on the effectiveness of the initial course of treatment. In a preferred embodiment, the initial treatment is limited to a single intravitreal injection of the VEGF antagonist. It is preferred that the VEGF antagonist is administered as a monotherapy (i.e. without coadministration of a further therapy such as laser photocoagulation).

Performing additional injections on an “as needed” basis reduces the total number of injections and thus decreases the risk of potential adverse events, e.g. due to general anaesthesia that may be needed for safe administration of the antagonist to infants.

In some cases, a single injection of the VEGF antagonist according to the invention may be sufficient to ameliorate the disease or prevent disease progression. In other cases, one injection is administered to the patient, and the need for one or more additional injection(s) is assessed at 4-16 weeks post injection. Re-treatment earlier than 4 weeks after the initial injection typically is to be avoided to prevent an increase in systemic exposure due to accumulation of intravitreal VEGF antagonist. If several additional injections are required, these additional injections also need to be administered at least 4 weeks apart. Treatment may be discontinued when all signs of retinal neovascularisation have disappeared completely. For example, treatment may be discontinued when no signs of recurrence of retinal neovascularisation can be observed for at least 12-24 weeks, e.g. 16 weeks. In particular, treatment is discontinued if there is no recurrence of ROP at 54 weeks of postmenstrual age.

Administration in an individualised “as needed” regimen is based on the treating physician's judgment of lesion/disease activity as assessed by the regression of retinal neovascularisation over time from baseline (i.e. after the initial dose of VEGF antagonist has been administered), e.g. starting at 4 weeks, and up to 12 months. For example, the VEGF antagonist is administered to an infant the first time after an initial diagnosis of a retinal neovascular disorder has been made. A diagnosis of a retinal neovascular disorder such as ROP can be made during examination of the eye by an ophthalmoscopy.

Ideally, lesion/disease activity is assessed weekly at least once after the initial dose and up to 16 weeks thereafter, followed by monthly reassessment for up to 12 months. Initial signs of a response to VEGF antagonist therapy can be observed as early as 7 days after the first injection, and therefore early assessment at day 7 after administration of the initial dose will provide an opportunity for early retreatment, if no signs of reduction in lesion/disease activity are observed. For example, if an initial dose of 0.06 mg or 0.075 mg intravitreal ranibizumab does not result in any reduction, a further injection of the same dose or a higher dose (e.g. twice or three times the initial dose) may be administered as early as 7 days after the first injection.

More commonly, disease activity will be assessed every 4-6 weeks after the initial administration of the VEGF antagonist. A second, third or further administration of the VEGF antagonist is performed only if examination of the eye reveals signs of a persistent or recurring retinal neovascular disorder, in particular ROP. Disease activity parameters (such as active angiogenesis, exudation and vascular leakage characteristics) are assessed by the change from baseline in anatomical endpoints over time starting from baseline (i.e. after the initial dose of VEGF antagonist has been administered), e.g starting at 4 weeks, and up to 12 months.

Further administrations of a VEGF antagonist is not deemed necessary if there is a regression of retinal neovascularisation, e.g. if the number of newly formed blood vessels at the follow-up visit is reduced from the number of newly formed blood vessels observed at baseline. If retinal neovascularisation recurs, no regression of new blood vessel formation is observed, or regression is deemed to be insufficient to prevent further damage to retina, a second, third of further administration of the VEGF antagonist is performed.

### Combination Therapy

The compounds of the invention may be administered in combination with one or more additional treatment(s), particularly if the patient does not respond to VEGF antagonist monotherapy.

Administration of the additional treatment (e.g. LPT or cryotherapy) and the VEGF antagonist should not occur simultaneously, so one will precede the other. The initiation of the additional treatment and of VEGF antagonist administration may occur within 2 and 24 weeks, e.g., within 4, 8 or 16 weeks of each other. Typically, VEGF antagonist therapy is administered prior to the additional treatment.

In some instances, the additional treatment is administered as needed. For example, the additional treatment may be performed only if examination of the eye reveals signs of persistent or recurring retinal neovascularisation after one or more (e.g. two, three or four) administrations of a VEGF antagonist. Disease activity parameters (such as active angiogenesis, exudation and vascular leakage characteristics) are assessed by the change from baseline in anatomical endpoints over time starting from baseline (i.e. after the initial dose of VEGF antagonist has been administered), e.g. starting at 4 weeks, and up to 12 months, as described above.

For example, the additional treatment may be administered if disease activity persists or worsens at 4 weeks, 6 weeks, 12 weeks, or 16 weeks after the initial administration of the VEGF antagonist. A worsening of disease activity is observed if the number of newly formed blood vessels at the time of assessment has increased over baseline (i.e. after the initial dose of VEGF antagonist has been administered).

In some instances, an additional treatment is administered prior to administration of a VEGF antagonist. For example, an additional treatment such as cryoretinopexy, sclera buckling or vitrectomy may be administered first when retinal detachment has occurred in order to prevent vision loss. VEGF antagonist therapy is administered subsequent to the additional treatment to prevent recurrence of retinal neovascularisation or detachment of the retina.

In one aspect of the invention, treatment with a VEGF antagonist of the invention may be used in combination with LPT as the additional treatment.

LPT uses laser light to cause controlled damage of the retina to produce a beneficial therapeutic effect. Small bursts of laser light can seal leaky blood vessels, destroy abnormal blood vessels, seal retinal tears, or destroy abnormal tissue in the back of the eye. LPT techniques and apparatuses are readily available to ophthalmologists (Lock et al. (2010) Med J Malaysia 65:88-94).

Panretinal LPT is typically used to stop neovascularisation in ROP infants by scattering burns throughout the peripheral retina. Laser spot sizes (spot diameters) of 50-500 μm are typical, applied for 50-200 ms, using green-to-yellow wavelengths e.g. using an argon gas (514.5 nm) laser, a krypton yellow laser (568.2 mn), or a tunable dye laser (variable wavelength). In some cases a red laser may be used if a green or yellow laser is precluded (e.g. if vitreous hemorrhage is present).

In a second aspect of the invention, treatment with a VEGF antagonist of the invention may be used in combination with cryotherapy as the additional treatment.

Cryotherapy is used to freeze and scar the peripheral retina thereby stopping abnormal blood vessel growth. During cryotherapy, a metal probe that has been exposed to a cryogen (typically liquid nitrogen) is placed on the sclera.

### General

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

### DESCRIPTION OF THE DRAWINGS

FIG. 1: Predicted exposure ratios of the maximum serum concentration (Cmax) of ranibizumab in infants receiving single bilateral intravitreal ranibizumab doses of 0.03-0.3 mg relative to the reference in vitro IC₅₀=11 ng/ml. Predicted ranges of exposure represent uncertainty in model assumptions.

FIG. 2: Predicted exposure ratios for the area under the curve (AUC) of ranibizumab in the serum (black) and vitreous (grey) of infants receiving single bilateral intravitreal ranibizumab doses of 0.03-0.3 mg relative to the reference AUC of ranibizumab in the serum of adults receiving a single unilateral intravitreal ranibizumab dose of 0.5 mg. Predicted ranges of exposure represent uncertainty in model assumptions.

FIG. 3: Predicted exposure ratios for the AUC of ranibizumab in the serum (black) and vitreous (grey) of infants receiving single bilateral intravitreal ranibizumab doses of 0.03-03 mg relative to the reference AUC of bevacizumab in the serum of infants receiving a single bilateral intravitreal ranibizumab dose of 0.625 mg. Predicted ranges of exposure represent uncertainty in model assumptions.

### MODES FOR CARRYING OUT THE INVENTION

### EXAMPLE 1

A Pharmacokinetic Model Jar Predictincr the Ocular and Systemic Exposure to Intravitreally Administered Ranibizumab in Infants

To model the ocular and systemic exposure to ranibizumab and bevacizumab in infants, two key relationships were established based on published data:
1. A relationship between the age of a child and the vitreous chamber depth and density of the vitreal gel to predict the ocular clearance rate and vitreal concentration;
2. A relationship between age and body weight of a child, and PK parameters of systemic disposition (allometric scaling) to predict the systemic concentration.

Vitreal concentration of ranibizumab and bevacizumab was calculated using the volume of the vitreous body. It was calculated as the volume of a partial sphere whose height equals the vitreous chamber depth (VCD) and whose diameter equals the axial length (AL) of the eye. The VCD and AL of premature infants at 10 weeks after birth was correlated with the infant birth weight using a linear regression model and published data (Fledelius (1992) Acta Ophthahnol Suppl 204:10-15). The VCD and AL of adults was age-correlated using a linear regression model and published data for adults (Neelam et al. (2006) Vision Res 46(13):2149-2156). The AL of the eye was calculated using an aspect ratio equal to the ratio of the average AL and VCD values obtained from the publications cited above.

Ocular clearance rate of ranibizumab and bevacizumab in the human eye was calculated using a one-dimensional model of diffusion and convection in a porous medium (Zhao & Nehorai (2006) IEEE Trans Signal Process 54(6):2213-2225; Dechadilok & Deen (2006) Ind Eng Chem Res 45(21):6953-6959). In this model, the eye is represented as a cylinder whose axis of symmetry coincides with the posterior-anterior axis of the eye. The front side of the cylinder is the hyaloid membrane next to the anterior chamber, and the back side of the cylinder is the retina. The length of the cylinder equals the VCD. In addition to the VCD, the ocular clearance rate in this model is determined by the density of the vitreal gel. A relationship between vitreal density and ocular clearance rate was established using published data (Tan et al. (2011) Invest Ophthalmol Vis Sci, 52(2):1111-1118). The relationship between age and vitreal density was based on published information (Oyster (1999) The Human Eye, Sinauer Associates Incorporated, pp. 530-544). The model was further calibrated to match the ocular kinetics established in adults for intravitreally administered ranibizumab and bevacizumab (the Novartis population PK model of ranibizumab and Zhu et al. (2008) Ophthalmology 115(10):1750-1755).

Systemic disposition of ranibizumab and bevacizumab was described using population PK models established for each of the respective antibody VEGF antagonists (the Novartis population PK model of ranibizumab and Lu et al. (2008) Cancer Chemother Pharmacol 62(5):779-786). Systemic bioavailability of bevacizumab was estimated using published data (U.S. Federal Drug Administration (2004) Review and Evaluation of Toxicology Data: Bevacizumab (Avastin), BLA STN#125085; Bakri et al (2007) Ophthalmology 114(5):855-859). The relationship between body weight and systemic clearance was modelled using standard allometric scaling principles (Anderson & Holford (2008) Annu Rev Pharmacal Toxicol 48(1):303-332). The relationship between age and body weight of premature infants at the time of the intravitreal injection was calculated using data on the distribution of body weight at birth (U.S. Center for Disease Control (2010) http://www.cdc.gov/nchs/VitalStats.htm) and a post-natal growth curve equation (Riddle et al (2006) Perinatol 26(6):354-358). The body weight of adults was calculated using established relationships between age and parameters of the body weight distribution (Portier et al (2007) Risk Anal 27(1):11-26).

Model simulations were performed for typical patients and provided an expected average exposure. A typical premature infant was modelled to be born at age 24.2 weeks (post-menstrual age) with a body weight of 929 g. The model further assumed that the infant was intravitreally injected with ranibizumab or bevacizumab at 34.5 weeks (post-menstrual age) to treat ROP, and that the infant had a body weight of 2092 g at the time of the injection, based on a typical growth curve. A typical adult was modelled to be 70 years old.

Exposure was simulated for a range of those key model parameters which are expected to impact the predicted exposure the most. Exponents of allometric scaling relationships between systemic clearance and volume of distribution and body weight were varied between 0.37-0.75 (clearance) and 0.41-1 (volume). Potentially greater permeability of the immature ocular membranes in young children was captured by increasing the ocular clearance rate by 50% relative to the adult value. Systemic bioavailability of intravitreally injected bevacizumab was varied between 0.65 and 0.92 (average value 0.77).

### EXAMPLE 2

Ranibizumab Dose Determination for Treating Infants with ROP

Using the pharmacokinetic model described in Example 1, the predicted ocular and systemic exposure in infants receiving intravitreally administered ranibizumab was compared to the exposure in adults following intravitreal injection of 0.5 mg ranibizumab, since the efficacy and safety profiles for adults at this dose level and mode of administration are known.

Exposure ratios to ranibizumab were calculated for three different parameters: (i) the maximum concentration (Cmax) in serum, which provides a measure of acute toxicity, (ii) the area under the curve (AUC) in serum, which provides a measure of potential long-term toxicity associated with continual inhibition of systemic VEGF, and (iii) the AUC in the vitreous which provides a measure of efficacy associated with continual inhibition of VEGF in the eye.

The ratio of predicted exposure in infants to exposure in adults represents a measure of likelihood of ocular and systemic toxicity and can be used to determine the relative benefit/risk ratio of paediatric doses. Doses with a systemic exposure ratio that are equal to or less than 1 are considered to have an acceptable safety profile. The serum concentration should also be lower than the in vitro IC₅₀ for ranibizumab which is in the range of 11-27 ng/ml. Doses with a vitreous exposure ratio close to 1 are considered to have an acceptable efficacy profile.

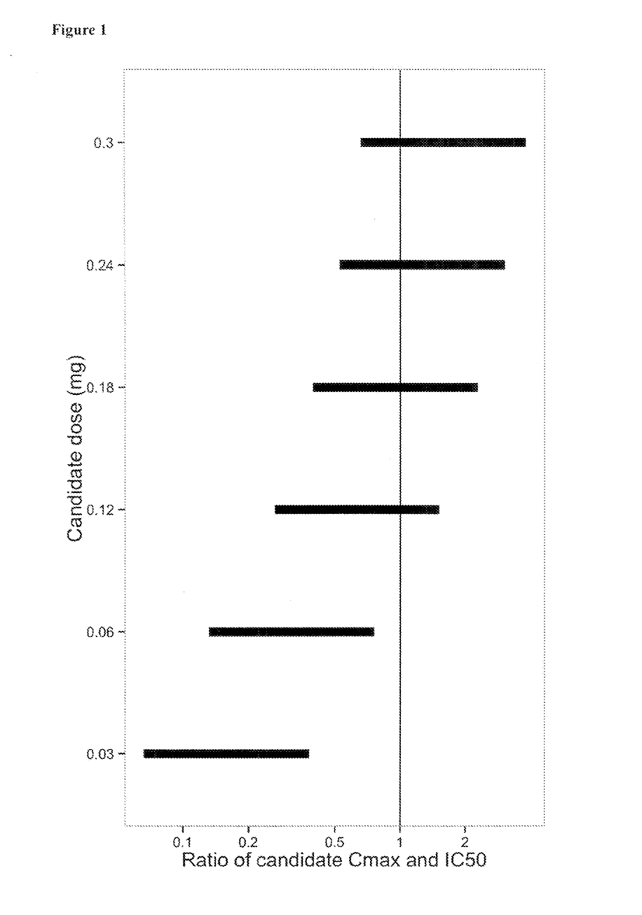

The predicted maximum concentration in serum (Cmax) was similar to the in vitro IC₅₀ for ranibizumab in infants at doses lower than 0.3 mg. However, based on the exposure ratio for Cmax to IC₅₀ in serum, a dose of less than 0.24 mg is preferable. A dose of 0.06 mg is even more preferable as only then the ratio of candidate Cmax and IC₅₀ is <1 (see FIG. 1).

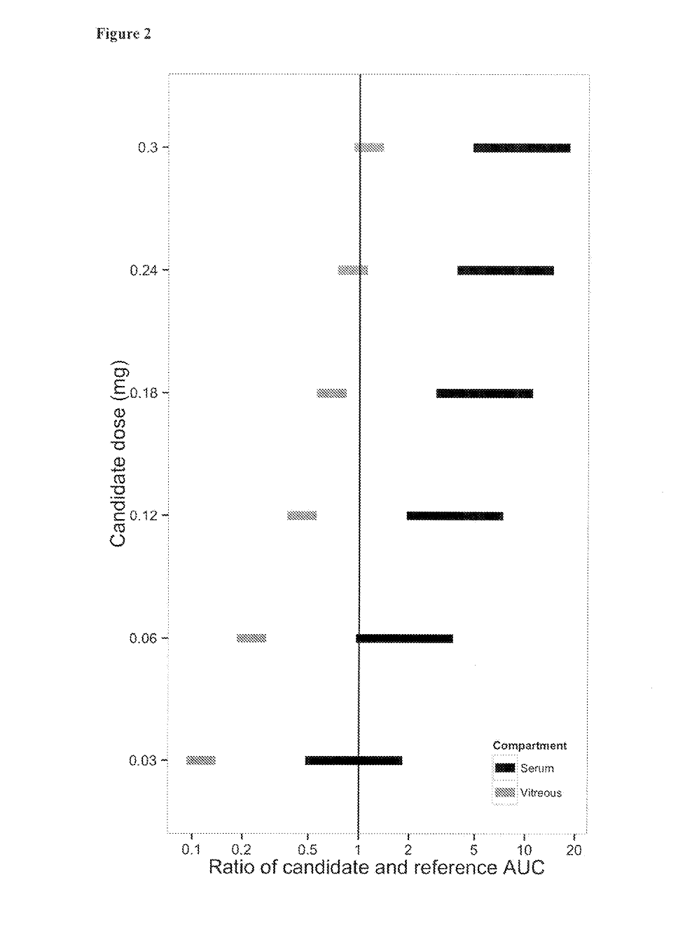

Exposure ratios of AUC in serum are greater than 1 for all modelled paediatric doses, while exposure ratios of AUC in vitreous are less than 1 (FIG. 2). When choosing specific doses for the administration to infants, the possibility of underexposure relative to the reference adult vitreal exposure (decreased efficacy) needs to be balanced against the increased serum AUC (increased risk). However, since the doses considered in the model hover around the in vitro IC₅₀ for ranibizumab, doses of up to 0.3 mg are deemed to have an overall acceptable safety profile, while approaching exposure levels in the vitreous that have been shown to be efficacious in adults. This suggests that all these doses have an appropriate benefit-risk profile. Therefore escalation to doses higher than 0.06 mg (i.e. up to 0.3 mg) may take place in context of a clinical study in case of insufficient efficacy at lower doses and absence of safety signals.

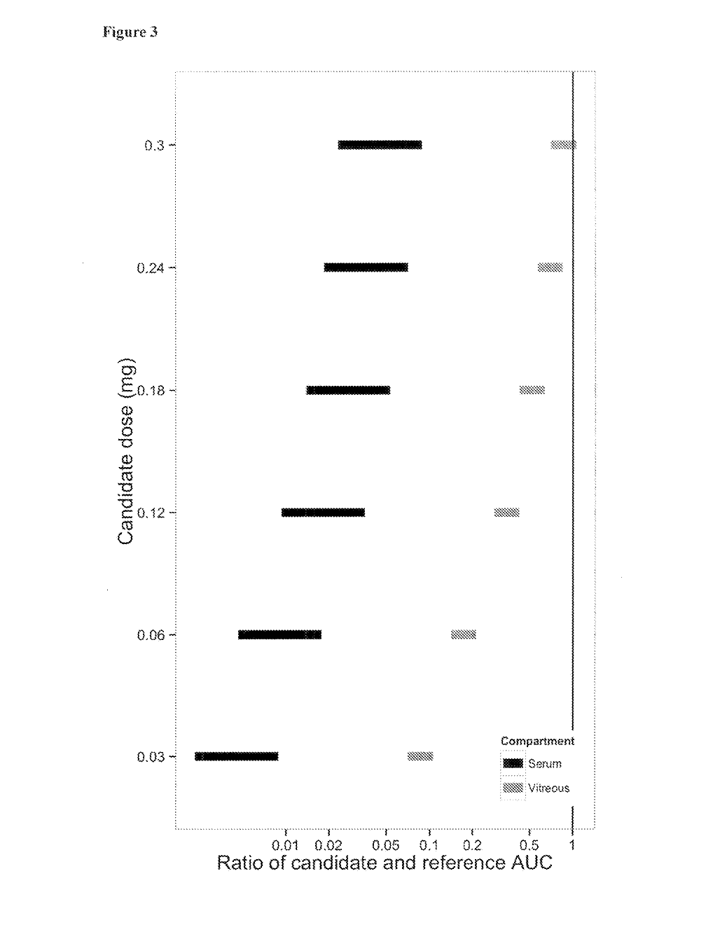

Importantly, the serum AUC for all ranibizumab doses in infants is much less than the serum AUC for 0.625 mg intravitreal bevacizumab used to treat ROP in the BEAT-ROP study (FIG. 3). This indicates a lesser extent of systemic VECF suppression than the reference prior art treatment while achieving comparable efficacy.

Dose adjustment for VEGF antagonists other than ranibizumab for the treatment of infants can be determined using the predicted ocular and systemic exposure data of ranibizumab described herein.

### EXAMPLE 3

Clinical Study to Determine the Effects of Two Different Doses of Intravitreal Ranibizumab in Infants with ROP

The proposed study will investigate in a 16-week post-injection study period whether (i) ranibizumab offers similar treatment effects in ROP as reported in the BEAT-ROP study for bevacizumab; (ii) lower doses of ranibizumab can achieve similar results in controlling ROP and (iii) if the two doses of ranibizumab differ in their effect on systemic VEGF suppression. This first period is followed by a non-interventional time frame of 5 years during which the treated infants will be assessed twice (at 2 and 5 years) for long-term ophthalmological and pediatric development including targeted examination of VEGF depending organs like heart, lungs, vascular system and brain.

40 ROP infants requiring treatment according to the current guidelines of the German ophthalmic society for ROP in zone II (stage 1+, 2+, 3+/− or AP-ROP) or central zone II (stage 3+) will be enrolled in this study. Treatment criteria have to be evaluated independently by two ophthalmologists experienced in screening and treating ROP. Only infants where both ophthalmologists independently agree on ROP staging and treatment are enrolled. Infants are randomized 1:1 to one of the two treatments arms.

Infants in treatment arm 1 receive a single intravitreal injection of 0.06 mg ranibizumab at day 0. Infants in treatment arm 2 receive a single intravitreal injection of 0.18 mg ranibizumab at day 0. A standard 6 mg/ml solution will be used to administer the respective doses.

Intravitreal injections will be performed by an ophthalmologist blinded to the content of the syringes used to administer ranibizumab. The treating ophthalmologist will receive two sterile syringes (0.1-0.2 ml per syringe, unlabelled)—one syringe for the left eye, one for the right eye.

Infants in both treatment arms will undergo anterior segment exam and fundoscopy for signs of treatment-associated immediate local complications (endophthalmitis, lens damage, retinal detachment, bleeding, media opacities, insufficient treatment response) and neonatal examination for signs of treatment-associated immediate systemic complications at days 1 and 3.

At day 7 and weekly at week 2-16, infants in both treatment arms will undergo anterior segment exam, fundoscopy and staging of ROP as well as neonatal examination.

Primary efficacy endpoint is the recurrence of retinopathy of prematurity in one or both eyes requiring re-treatment before 54 weeks' postmenstrual age. Re-treatment will consist of either bevacizumab injection or laser photocoagulation according to the current recommendation of the German Retinal Society (RG), the Federal Association of German Ophthalmologists (BVA) and the German Ophthalmological Society (DOG).

As secondary endpoint, the change in VEGF levels in peripheral blood over the first 16 weeks after intravitreal injection will be measured. VEGF will be measured by an ELISA-based test for human VEGF-A once prior to injection and then 1 week, 2 weeks, 4 weeks, 6 weeks, 8 weeks, 12 weeks and 16 weeks after intravitreal injection.

Non-interventional study phase from 16 weeks after injection until 2 years and 5 years of age includes assessment of the ophtalmological development (visual acuity, orthoptic status, cycloplegic retinoscopy, slit lamp exam, IOP, fundoscopy, OCT, fundus photographs and mfERG) and the pediatric development (developmental milestones, weight, height, cognitive, motor and sensory development).

### EXAMPLE 4

Comparing Alternative Ranibizumab Dosages for Safety and Efficacy Retinopathy of Prematurity (CARE-ROP)

This study is designed as an exploratory study to assess safety and efficacy of two different doses of the anti-VEGF agent ranibizumab (0.12 mg vs. 0.20 mg) in the treatment of infants with retinopathy of prematurity.

40 infants diagnosed with bilateral Retinopathy of Prematurity (ROP) in zone I (stage 1+, 2+, 3+/−, AP-ROP) or ROP in central (=posterior) zone II (stage 3+, AP-ROP) are enrolled in this study. Zone I is defined as twice the distance from the optic disc to the fovea measured temporally, posterior zone II is defined as three times the distance from the optic disc to the fovea measured temporally.

Infants are not included in the treatment if (i) there are pediatric conditions rendering the infant ineligible to anti-VEGF treatment or to repeated blood draws as evaluated by a neonatal ICU specialist and a study ophthalmologist; (ii) there are congenital brain lesions significantly impairing optic nerve function; (iii) there is severe hydrocephalus with significantly increased intracranial pressure; (iv) they have advanced stages of ROP with partial or complete retinal detachment (ROP stage 4 and 5); (v) there is ROP involving only the peripheral retina (i.e. peripheral zone II or zone III); (vi) there is known hypersensitivity to the study drug or to drugs with similar chemical structures; (vii) there are contraindications for an intravitreal injection as listed in ranibizumab SmPC; (viii) there is systemic use of anti-VEGF therapeutics; (ix) there is use of other investigational drugs—excluding vitamins and minerals—at the time of enrollment, or within 30 days or 5 half-lives prior to enrollment, whichever is longer.

Infants are randomized to one of the two treatments arms:
(1) Infants in treatment arm 1 receive an intravitreal injection of 0.12 mg (20 μl of the 6 mg/ml) ranibizumab at day 0. After an initial response the same dose as in the first injection can be re-applied after at least four weeks post injection. A maximum number of 3 regular re-injections can be applied.
(2) Infants in treatment arm 2 receive an intravitreal injection of 0.20 mg (20 μl of the 10 mg/ml) ranibizumab at day 0. After an initial response the same dose as in the first injection can be re-applied after at least four weeks post injection. A maximum number of 3 re-injections can be applied.

Primary outcome is the efficacy of treatment. Efficacy is determined by the number of infants without need for rescue treatment op to week 24 post first injection. Re-injection of study dose is not considered rescue treatment if applied after an initial response to treatment and after at least 4 weeks post injection. Secondary outcome includes (i) regression of plus disease; (ii) regression of preretinal vascularized ridge; (iii) progression of peripheral intraretinal vascularization beyond ridge; (iv) number and kind of AEs and SAEs; (v) changes in vascular endothelial growth factor (VEGF) levels in the systemic circulation; (vi) number of re-injections of study dose; (vii) number of patients progressing to stage 4 or 5 ROP; (viii) number of patients with complete vascularization of the peripheral retina to within one disc diameter of the ora serrate. Secondary outcomes are measured within a time frame of up to 24 weeks post first injection. In addition, other outcome measures include number of late recurrences of ROP during the follow-up period; number of patients progressing to stage 4 or 5 ROP after the core study; number of patients with complete vascularization of the peripheral retina to within one disc diameter of the ora serrata after the end of the core study; long-term ophthalmological development: visual acuity (if possible), orthoptic status, cycloplegic retinoscopy, refraction, IOP, fundoscopy including fundus photographs (at one year and at 5 years an ophthalmological visit will take place); long-term pediatric development: Bayley-test, weight, height, cognitive, motor and sensory development; number and kind of AES or SAEs per group between the end of the observational core study and the end of the follow-up period. Additional outcomes are measured within a time frame of up to 5 years post first injection.

### EXAMPLE 5

This is an open-label, randomized, parallel-group superiority study evaluating the efficacy and safety of ranibizumab 0.1 mg, ranibizumab 0.2 mg and laser therapy for the treatment of retinopathy of prematurity (ROP).

The aim of the study is to demonstrate that ranibizumab has superior efficacy compared to standard of care laser therapy as assessed by the proportion of patients in each treatment arm with the absence of active ROP and unfavorable structural outcomes 24 weeks after first treatment.

The primary objective is to test for superiority of the 0.2 mg ranibizumab dose against laser therapy. Key secondary objectives are to test for superiority of the 0.1 mg ranibizumab dose against laser and to test for superiority of the 0.2 mg ranibizumab dose against the 0.1 mg ranibizumab dose. Ocular and systemic safety of the treatments will also be assessed.

The study comprises three arms, each of 80 patients. The patients are male and female premature neonates with bilateral ROT requiring treatment.

Group 1: A single intravitreal injection of 0.1 mg ranibizumab (10 mg/ml) to each eye at baseline.

Group 2: A single intravitreal injection of 0.2 mg ranibizumab (10 mg/ml) to each eye at baseline.

Group 3: Control. Laser photocoagulation therapy to each eye at baseline.

Secondary endpoints are proportion of patients at 24 weeks after starting study treatment, who: required rescue treatment, have absence of active ROP, have absence of unfavorable structural outcome, or require 1, 2 or 3 ranibizumab re-treatments.

The incidence of ocular and systemic adverse events is evaluated at 24 weeks. The disease recurrence rate is evaluated at 24 weeks. The time to first recurrence of ROP in each treatment arm is measured up to 24 weeks.

Laser photocoagulation may be used as a rescue treatment if patients do not respond to ranibizumab treatment.

### EXAMPLE 7

The purpose of this study is to report the management of infants treated with anti-VEGF at the Jules Stein Eye Institute/University of California, Los Angles (UCLA) (Wong, Ryan K.; Tsui, Irena, ARVO 2014).

### Method

A retrospective chart review was conducted on consecutive infants screened for retinopathy of prematurity in the neonatal intensive care unit at the Ronald Regan UCLA Medical Center from January 2012 to December 2013. Babies with type 1 prethreshold disease or worse and treated with anti-VEGF therapy were identified. All infants with at least 6 months of follow up at time of publication were included.

### Results

Six eyes (4 out-born infants) were included in the study. The mean birth weight was 605 grams (range: 500-690 grams), mean gestational age was 23.4 weeks (range: 23.0-24.3 weeks), and mean age at time of anti-VECF injection (4 eye with ranibizumab (0.25 mg) and 2 eyes with bevacizumab (0.625 mg)) was 34.2 weeks (range: 31.6-36.3 weeks). All eyes had stage 2 or 3 Retinopathy of Prematurity (ROP), posterior zone 2, with plus disease. All eyes showed initial resolution of plus disease and regression of ROP after treatment. All 6 eyes required additional laser treatment, at a mean age of 44.4 weeks (range: 42.9-50.4 weeks). Indications for additional laser were reactivation of ROP in 3 eyes (50%), at an average of 6.1 weeks after anti-VEGF treatment, and persistent stage 1, zone 3 ROP in 3 eyes (50%), at an average of 12.9 weeks after anti-VEGF treatment.

### Conclusions

Infants after anti-VEGF therapy for ROP may often require supplemental laser photocoagulation for reactivation or persistence of disease within 3 months.

It will be understood that the invention has been described by way of example only and modifications may be made whilst remaining within the scope and spirit of the invention.

1. A method for treating an infant having a retinal neovascular disorder comprising administering to an eye of said infant a VEGF antagonist that either does not enter or is rapidly cleared from the infant's systemic circulation.

2. The method of claim 1, wherein the VEGF antagonist is ranibizumab.

3. The method of claim 1, wherein the VEGF antagonist is a non-antibody VEGF antagonist.

4. The method of claim 3, wherein the non-antibody VEGF antagonist is selected from a recombinant human soluble VEGF receptor fusion protein and a recombinant binding protein comprising an ankyrin repeat domain that binds VEGF-A.

5. The method of claim 3, wherein the non-antibody VEGF antagonist is a small-molecule compound.

6. The method of claim 1, wherein the retinal neovascular disorder is secondary to retinopathy of prematurity (ROP).

7. The method of claim 1, wherein the VEGF antagonist is administered at dose that is less than 50% of the dose typically administered to an adult receiving treatment for a retinal neovascular disorder.

8. The method of claim 7, wherein the dose is less than 30% of the dose typically administered to an adult receiving treatment for a retinal neovascular disorder.

9. The method of claim 1, wherein the VEGF antagonist is administered in a volume that is less than 50% of the volume typically administered to an adult receiving treatment for a retinal neovascular disorder.

10. The method of claim 9, wherein the volume is less than 30% of the volume typically administered to an adult receiving treatment for a retinal neovascular disorder.

11. The method of claim 2, wherein the dose of ranibizumab administered to the infant is 0.05-0.25 mg, preferably 0.1-0.2 mg.

12. The method of claim 11, wherein the dose of ranibizumab administered to the infant is selected from the group consisting of 0.06 mg in 10 μl, 0.075 mg in 7.5 μl, 0.1 mg in 10 μl, 0.12 mg in 20 μl, 0.15 mg in 15 μl, 0.18 mg in 30 μl, 0.20 mg in 20 μl, 0.25 mg in 25 μl, and 0.24 mg in 40 μl.

13. The method of claim 1 comprising administering a first dose of the VEGF antagonist, wherein a second dose of the VEGF antagonist is administered as needed but at least 7 days, more preferably 4 weeks, after the first injection.

14. The method of claim 13, wherein the first dose and the second dose are at least 16 weeks apart.

15. The method of claim 13, wherein the second dose is administered when no regression of retinal neovascularisation is observed after administration of the first dose, or when the treating physician deems retinal neovascularisation to have regressed insufficiently to prevent damage to the infant's retina in the treated eye.

16. The method of claim 13, wherein the second dose is administered if retinal neovascularisation recurs after having regressed subsequent to administration of the first dose.

17. The method of claim 1, wherein the method further comprises administering laser photocoagulation therapy (LPT) or cryotherapy.

18. The method of claim 17, wherein initiation of LPT or cryotherapy and initiation of VEGF antagonist administration occur within 2 and 24 weeks of each other.

19. The method of claim 17, wherein initiation of the VEGF antagonist administration occurs before LPT or cryotherapy.

20. The method of claim 19, wherein LPT or cryotherapy is performed only if examination of the treated eye reveals signs of persistent or recurring retinal neovascularisation.
